(19) United States
(12) Patent Application Publication
(10) Pub. No.: US 2007/0155009 A1
(43) Pub. Date: Jul. 5, 2007

(54) EXTRACELLULAR MATRIX COMPONENTS FOR EXPANSION OR DIFFERENTIATION OF HEPATIC PROGENITORS

(75) Inventors: Randall E. McClelland, Chapel Hill, NC (US); Lola M. Reid, Chapel Hill, NC (US)

Correspondence Address: FOLEY AND LARDNER LLP;SUITE 500, 3000 K STREET NW, WASHINGTON, DC, 20007, US

(73) Assignee: UNIVERSITY OF NORTH CAROLINA AT CHAPEL HILL

(21) Appl. No.: 11/560,049

(22) Filed: Nov. 15, 2006

Related U.S. Application Data
(60) Provisional application No. 60/736,873, filed on Nov. 16, 2005.

(51) Int. Cl. C12N 5/06; C12N 5/08; C12M 3/00

(52) U.S. Cl. 435/325; 435/370; 435/289.1

(57) ABSTRACT

A method is provided of propagating hepatic progenitors in vitro on or in one or multiple extracellular matrix components found in the stem cell compartment or niche of liver. A container for the propagation of the progenitors and comprising culture dishes, bioreactors, or lab chips.

### CROSS REFERENCES TO RELATED APPLICATIONS

This application claims priority to U.S. Provisional Application No. 60/736,873, filed Nov. 16, 2005, the disclosure of which is incorporated by reference herein in its entirety.

### FIELD OF THE INVENTION

The present invention relates generally to the ex vivo propagation or differentiation of hepatic progenitor cells. More particularly, the present invention relates to the identification and selection of extracellular matrix components, which enable the propagation and/or differentiation of hepatic progenitor cells, including hepatic stem cells, in vitro.

### BACKGROUND OF THE INVENTION

Hepatic stem cells and their progeny (e.g., hepatoblasts and committed progenitors) have considerable expansion potential. For this reason, these cell populations are desirable candidates for cell therapies, including bioartificial livers or cell transplantation. Despite this promise, however, the full potential of liver cell therapy remains to be realized.

In part, the ex vivo propagation of hepatic stem cells and their progeny has proven to be challenging. Where hepatic stem cells and their progeny are successfully propagated in vitro, the culture conditions are not optimal for transition from the laboratory bench to the clinic. For example, some culture conditions greatly retard cell division or arbitrarily promote cell differentiation, thereby reducing propagation efficacy. As well, some culture conditions require the addition of factors (e.g., serum or feeder cells) that can introduce contaminants and thereby limit their application in treating humans.

Accordingly, there is a need for culture conditions that can provide enhanced ex vivo propagation of hepatic stem cells and their progeny and a need for conditions that can lineage restrict stem cells into their appropriate fates. In addition, there is a need for culture conditions that obviate the heretofore requirement of feeder cells.

### SUMMARY OF THE INVENTION

In one embodiment of the present invention, a method of propagating hepatic progenitors in vitro is provided, comprising: (a) providing isolated hepatic progenitors and (b) culturing the isolated hepatic progenitors on a layer comprising one or a combination of extracellular matrix components found in the stem cell compartment of liver. The extracellular matrix component(s) may be a type III collagen, a type IV collagen, a laminin, hyaluronans, other glycosaminoglycans, heparan sulfate proteoglycans, chondroitin sulfate proteoglycans or a combination thereof. In some embodiments, the layer may comprise other extracellular matrix component, which can be proteoglycans such as agrin, perlecan, integrin, nidogen, dystroglycan, or others, or basal adhesion molecules such as a fibronectin, or other proteins such as elastin and combinations thereof. In a preferred embodiment of the instant invention, the first extracellular matrix component is type III collagen and the second extracellular matrix protein is laminin.

According to the inventive method, the isolated hepatic progenitors may be isolated hepatic stem cells, isolated hepatoblasts, committed hepatic progenitors, or a combination thereof. As well, the method may further comprise culturing the hepatic progenitors in the presence of feeder cells, which cells may be embryonic, fetal, neonatal and/or murine in origin. Preferably, the feeder cells are angioblasts. The method may yet further comprise culturing the hepatic progenitors in a serum-free culture medium. Preferably the serum-free medium contains insulin (5 μg/ml), transferrin/fe (5 μg/ml), and a mixture of lipids (free fatty acids, high density lipoproteins), low calcium (<0.5 mM), and little or no copper. The hepatic progenitors may be obtained from fetal, neonatal, pediatric or adult livers.

The laminin may be at a concentration between about 0.1 to about 10 μg/cm², preferably between about 0.5 to about 5 μg/cm², and more preferably at a concentration of about 0.5 μg/cm² or 1 μg/cm². The type III or IV collagens are individually at a concentration between about 0.1 to about 15 μg/cm², preferably between about 0.5 to about 8 μg/cm², and most preferably between about 1 to about 7 μg/cm².

In another embodiment of the present invention, a method of propagating hepatic progenitors is provided, comprising: (a) providing a first layer comprising one or more extracellular matrix component(s) found in the stem cell compartment of liver; (b) providing a second layer comprising one or more extracellular matrix component (s) found in the stem cell compartment of liver; and (c) culturing isolated hepatic progenitors between the first and the second layers. The extracellular matrix component(s) may be a type III collagen, a type IV collagen, a laminin, hyaluronans, a proteoglycan (e.g., heparan sulfate and/or chondroitin sulfate proteglycan) or a combination thereof. In some embodiments, the layer may comprise other extracellular matrix components, which can be other proteoglycans (e.g., agrin, perlecan, nidogen, dystroglycan), other basal adhesion molecules (e.g., a fibronectin) and other extracellular matrix protein (e.g., elastin) and combinations thereof. In a preferred embodiment of the instant invention, the first extracellular matrix component is type III collagen and the second extracellular matrix component is laminin.

According to the inventive method, the isolated hepatic progenitors may be isolated hepatic stem cells, isolated hepatoblasts, committed hepatic progenitors, or a combination thereof. As well, the method may further comprise culturing the hepatic progenitors in the presence of mesenchymal progenitors, presented as feeder cells, which cells may be derived from embryonic, fetal, neonatal, pediatric or adult tissues and may be from any mammalian species. Preferably, the feeder cells are angioblasts. Preferably the angioblasts derive from liver. and preferably the angioblasts derive from a species identical to that of the hepatic progenitors. The method may yet further comprise culturing the hepatic progenitors in serum-free culture medium. The hepatic progenitors may be obtained from fetal, neonatal, pediatric or adult livers.

The laminin may be at a concentration between about 0.1 to about 10 μg/cm², preferably between about 0.5 to about 5 μg/cm², and more preferably at a concentration of about 0.5 μg/cm² or 1 μg/cm². The type III or IV collagens are individually at a concentration between about 0.1 to about 15 μg/cm², preferably between about 0.5 to about 8 μg/cm², and most preferably between about 1 to about 7 μg/cm².

In yet another embodiment of the present invention, A container for propagation of hepatic progenitors is provided, comprising: (a) a container (e.g., tissue culture plate, a lab chip, a bioreactor) and (b) an insoluble layer comprising at least one extracellular matrix component found in the stem cell compartment or niche of livers, wherein the insoluble material substantially coats a surface of or in the container.

As such, those skilled in the art will appreciate that the conception upon which this disclosure is based may readily be utilized as a basis for the designing of other structures, methods and systems for carrying out the several purposes of the present invention. It is important, therefore, that the claims be regarded as including such equivalent constructions insofar as they do not depart from the spirit and scope of the present invention.

### BRIEF DESCRIPTION OF THE DRAWINGS

FIG. 1 is a schematic of the culture design systems and collagen matrix substrata. A.) A control culture with hepatoblasts seeded on the tissue culture plastic (TCP) surface while supported with nutrient media. B.) Displays one ratio of the elements used to establish a collagen matrix substrate. C.) Is the Flat Plate design of collagen seeded with hepatoblasts. D.) Is a “Sandwich” design of collagen and hepatoblasts.

FIG. 2 is a schematic of the technique to apply a monolayer of matrix onto a TCP surface. A.) Illustrates the initial step utilized to precoat the surface. Matrix molecules are heterogeneously distributed onto the surface for a set time period. B.) The sterilization process. C.) Three 1×PBS washings to neutralize acetic pH that occur during procedures to establish collagen gels or fibrils. D.) Final product with liver cells seeded as a monolayer onto matrices and supported by nutrient media.

FIG. 3 provides photographs of immunohistochemistry results for matrix components found in STO 5 cultures.

FIG. 4 shows time-lapse 20× micrographs of liver cell characteristics for cultures days 0, 5, 15, and 31. FIG. 4A: day 0 showing hepatoblasts as large clumped cells along with interdispersed single cells. FIG. 4B: day 5 showing confluent hepatoblasts with associated mesenchymal cells. FIG. 4C: day 15 with equal amounts of hepatoblasts and mesenchymal cells. FIG. 4D: day 31 with abundance of mesenchymal cells.

FIG. 5 shows day 10, 10× micrographs of human fetal liver cells cultured within the control TCP, Flat Plate, and Sandwich design systems. A.) Control culture response—hepatoblasts (h) engulfed by mesenchymal (np) cells. B.) Flat Plate culture response—hepatoblast (h) colony with a few mesenchymal cells. C.) Sandwich culture response—hepatoblast (h) colony immunostained for albumin (red) and CK19 (green).

FIG. 6 shows urea production from cells grown on the various matrices. A.) Urea functions found in hepatoblasts cultured on collagen III (▪), collagen IV (●), laminin (∘), and a mixture of collagen IV-laminin [symbol] are analyzed and compared against plastic control (▾) cultures. B.) Urea functions for hepatoblasts cultured on TCP (●), Flat Plate collagen I (∘), and Sandwich Collagen I

[symbol]

FIG. 7 shows glucose and ammonia production from cells grown on the various matrices. A.) Glucose liver functions for hepatoblasts cultured on control TCP (▪), Flat Plate {grave over (()} ), and Sandwich [symbol] system designs. B.) Ammonia liver levels for hepatoblasts cultured on control TCP (▪), Flat Plate {grave over (()} ), and Sandwich [symbol] system designs.

FIG. 8 shows in vitro growth characteristics of liver progenitor cells grown on matrix components of the stem cell niche. 10-day culture images are displayed over the entire petri dish surface for hepatoblasts seeded on A1.) collagen III, B1.) laminin, and C1.) a mixture of collagen III and laminin. Comparatively, the same cultures are observed with 10× magnification (A2, B2, and C2) to illustrate cellular level responses.

FIG. 9 shows the outgrowth of hepatic stem cell colonies after seeding of fetal human liver cells on embryonic matrix substrates (collagen III and collagen IV). Day 3 hepatoblasts (h) are evident on all matrix substrates. Non-parenchymal cells (np) are also evident under all conditions and are labeled in the micrographs. Hepatic stem cells are found initially on day 3 in cultures on collagen III, but they appear also in day 10 cultures on TCP, collagen III, and collagen IV.

FIG. 10 shows Hepatic Stem Cells selected according to the instant invention. Differentiation dynamics in long-term cultures with eruptions of distinctive cell types, determined to be hepatoblasts, emerge from the edges of an HSC colony on day 12. The highlighted region in A) illustrates the HSC colony as it evolved from days 3-11, and B) displays the eruption within an 8-hour time period.

FIG. 11 provides a schematic process of purifying Livercell-stock into HSCcell-stock.

FIG. 12 provides a schematic of a passaging protocol: HSCs are initially seeded on control TCP dishes, as illustrated in A. Next, HSCs are passaged onto 0.4 μm porous inserts maintained in 6-well culture containers as shown in B. C is a top view of an untreated insert. D is the top view of a collagenIII precoated insert. E illustrates the final product of cell passaged onto insert surfaces.

FIG. 13: A1.) HSC aggregated surrounded by hepatoblasts seeded on TCP. A2.) Magnification view of HSC aggregate in A1. B1.) HSC aggregated after purification by ficol fractionation, seeded on TCP. B2.) Magnification view of HSC aggregate in B1. Informative table below micrographs displays immunofluorescent responses. Highly active (++), active (+), variable (+/−), and negative (−).

FIG. 14 shows immunoflourescent labeling for EpCAM, CK19, and NCAM. EpCAM is highly positive (A1 phase) vs. (A2 flourescent). Ck19 displays variability (B1 & C1 phase) vs. (B2 neg HSC fluorescence & C2 positive fluorescence). NCAM also displays variability (D1 & E1 phase) vs. (D2 positive & negative HSC fluorescence & E2 positive fluorescence).

FIG. 15 shows a comparison of number of HSC colonies formed when seeded on TCP or Bornstein and Traub (BoTr) collagen type-I fragment substrata (Sigma Type-III Collagen). Additionally, the detail illustrates colony formation on other adult (collagen type-I) and fetal (BD collagen III & IV) matrices.

FIG. 16 shows the response of HSC colony expansion with seeded on TCP or Bornstein and Traub (BoTr) collagen type-I fragment substrata (Sigma Type-III Collagen). Arrows point to newly visualized colonies on day 7. Day 18 displays large colony aggregates with the BoTr surface in this 4× micrograph. Day 30 shows disbanding aggregates for both seeding surfaces. 20× detail morphologies are shown for plastic, BoTr, and fetal collagen III & IV matrices.

FIG. 17 shows the total aggregate proliferation patterns for HSCs seeded on TCP (black) or Bornstein and Traub (BoTr) collagen type-I fragment substrata (Sigma Type-III Collagen (gray). For image normalization, the large circle patterns with “wavy” lines represent the total seeding surface (35 mmD dish). HSCs seeded on BoTr proliferate earlier (day 5), achieve larger surface coverage (day 20) and dissipate from the surface slower (day 30) than control. The detailed image compares colony growth when seeded on the different matrix substrates of collagen III & IV from BD and collagen I from Vitrogen.

FIG. 18 provides normalized cell quantities for days 5-30 illustrating variations in log phase growth (days 5-10) versus saturation density kinetics (days 10-20) versus post-confluence (days 20-30). HSC BoTr seeded cultures illustrate improved proliferation numbers throughout the culture periods.

FIG. 19 shows variations in log phase growth (days 5-10), saturation density kinetics (days 10-20), and post-confluence (days 20-30) display “Doubling” or “Waning” aspects of cell proliferation.

FIG. 20 shows the passaging of HSC onto 4 unique surfaces. 17 colonies are initially passaged. 14 days post passaging compares HSC colony attributes. Passaged HSCs on Bornstein and Traub (BoTr) collagen type-I fragment substrata (Sigma Type-III Collagen, 6 μg/cm²) provides the most efficient environment.

FIG. 21 shows normalized albumin function comparing hepatoblast (days 5-11), HSCs and hepatoblasts (days 9-17), and HSCs only (days 12-30).

FIG. 22 shows telomerase activity levels of Hepatic Stem Cells cultured on TCP (black bar) and Bornstein and Traub (BoTr) collagen type-I fragment substrata (Sigma Type-III Collagen) (light grey bar) at days 10 and 20—with HeLa cells (dark grey bar) as the “baseline comparison”. A.) displays activities for samples normalized to Protein Levels. B.) displays activities for samples normalized to Cell Numbers. Micrographs are the same Human HSCs in phase, negative control, and with telomerase expression.

### DETAILED DESCRIPTION OF THE INVENTION

In one embodiment of the present invention, extracellular matrix components have been identified, which facilitate the attachment, survival and ex vivo proliferation of hepatic stem cells and their progeny. The term “hepatic progenitors,” as used herein, is broadly defined to encompass both hepatic stem cells and their progeny. “Progeny” may include both self-replicating hepatic stem cells, hepatoblasts, pluripotent progenitors thereof, and progenitors committed to differentiate into a particular cell type (e.g., a hepatocyte).

“Clonogenic expansion” refers to the growth property of cells that can expand from a single cell and be subcultured and expanded repeatedly with retention of the phenotype of the parental cell. “Colony formation” refers to the property of diploid parenchymal cells that can undergo a limited number of cell divisions (typically 5-7 cell divisions) within a week or two and involves cells with limited ability to undergo subculture or passaging. “Pluripotent” signifies cells that can form daughter cells of more than one fate; “unipotent” or “committed progenitors” are cells that have a single adult fate.

Hepatic stem cells (HSCs) are pluripotent cells found in the ductal plates (also called limiting plates) in fetal and neonatal livers and in the Canals of Hering in pediatric and adult livers and showing evidence of self-replication (ref) with expression of telomerase and being capable of forming mature liver cells when transplanted (refs). These cells are EpCAM+, NCAM+, ALB+, CK8/18+, CK19+, CD133/1+, and are negative for all hemopoietic markers tested (e.g., CD34, CD38, CD45, CD14), mesenchymal cell markers (CD146, VEGFr, CD31) and for expression of P450s or alpha-fetoprotein. The HSCs have been found to give rise to hepatoblasts and to committed (unipotent) biliary progenitors.

Hepatoblasts (HBs) are pluripotent cells found throughout the parenchyma of fetal and neonatal livers and as single cells or small aggregates of cells tethered to the ends of the Canals of Hering. HBs derive from the HSCs. HBs share many antigens present on HSCs but with important distinctions. For example, HBs do not express NCAM but rather ICAM1 and they express significant amounts of alpha-fetoprotein and fetal forms of P450s. These HBs give rise to the unipotent progenitors, the committed hepatocytic and biliary progenitors.

Hepatic Committed Progenitors are unipotent progenitors of either the hepatocytic and biliary lineages. Their antigenic profile overlaps with that of the HBs; however, biliary committed progenitors express CK19 but not AFP or ALB, whereas the hepatocytic committed progenitors express AFP and ALB but not CK19. Committed biliary progenitors derive directly from hepatic stem cells and also from hepatoblasts.

Mesenchymal Cells (MCs) include cells at various lineage stages of the many different mesenchymal cell types (listed as the mature cells and, in parentheses, their precursors): including stroma (mesenchymal stem cells), endothelia (angioblasts), stellate cells (stellate cell precursors), and various hemopoietic cells (hemopoietic stem cells)

While most, if not all, of the discussion and examples of hepatic progenitors herein will be with reference to human-derived cell populations, the teachings herein should not be limited to humans. In fact, one of ordinary skill in the art may be expected to apply the teachings herein to the expansion of hepatic progenitors from mammals, generally (e.g., mice, rats, dogs, etc.) Accordingly, the scope of the present invention is intended to include hepatic progenitors of any and all mammals.

It is also noted that hepatic progenitors suitable for in vitro propagation in accordance with the instant invention are not limited to those isolated or identified by any particular method. By way of example, methods for the isolation and identification of the hepatic progenitors have been described in, for example, U.S. Pat. No. 6,069,005 and U.S. patent application Ser. Nos. 09/487,318; 10/135,700; and 10/387,547, the disclosures of which are incorporated herein in their entirety by reference.

Hepatic stem cells and hepatoblasts have characteristic antigenic profiles and can be isolated by protocols described previously. For example, hepatic stem cells and hepatoblasts share numerous antigens (e.g., cytokeratins 8, 18, and 19, albumin, CD133/1, and epithelial cell adhesion molecule (“EpCAM”) and are negative for hemopoietic markers (e.g., glycophorin A, CD34, CD38, CD45, CD14) and mesenchymal cell markers (e.g., CD146, CD31, VEGFr or KDR). Alternatively, hepatic stem cells and hepatoblasts can be distinguished from each other by size (the stem cells are 7-9 μm; the hepatoblasts are 10-12 μm), by morphology in cultures (the stem cells form dense, morphologically uniform colonies, whereas the hepatoblasts form cord-like structures interspersed by clear channels, presumptive canaliculi), by distinctions in the pattern of expression of certain antigens (EpCAM is expressed throughout the hepatic stem cells but is confined to the cell surface in the hepatoblasts), or by distinct antigenic profiles (N-CAM is present in the hepatic stem cells, whereas alpha-fetoprotein (AFP) and ICAM1 are expressed by the hepatoblasts). In fetal and neonatal livers, the hepatic stem cells are in the ductal plates (also called “limiting plates”), whereas the hepatoblasts are the dominant parenchymal cell population (>80%). In pediatric and adult tissues, the hepatic stem cells are present in the Canals of Hering, whereas the hepatoblasts are cells tethered to the ends of the Canals of Hering. The hepatoblasts consist of small numbers of cells in normal tissue but found in large numbers (e.g., nodules) in diseased livers (e.g., cirrhosis).

The present inventors have found that extracellular matrix components found in or near the liver's stem cell niche, provide for expansion of hepatic progenitors without inducing differentiation better than existing technology. As will be described in greater detail below, cells cultured on the matrix components, found in abundance in or near the liver's stem cell niche, aggregate to form spheroid-like structures on some of the matrix components (e.g., laminins) and spread into monolayers on others (e.g., type III collagen). Specific types of extracellular matrix components, found in the stem cell niche, are among the signals requisite for hepatic progenitor cells to undergo expansion in self-replication mode, that is symmetric cell divisions (the daughter cells are identical or nearly identical to the parent cells.

It is further believed that the maturation of hepatic stem cells occurs concomitantly with a unique combination of matrix components which direct, at least in part, their differentiation. Some extracellular matrix components are permissive for hepatic progenitors to undergo expansion associated with asymmetric divisions, that is expansion along with some differentiation. Yet others, located in regions of the liver tissue in which fully mature liver cells are found, elicit growth arrest and full differentiation of the cells.

Thus, maintenance of hepatic stem cells in their immature form in vitro is aided by their culture in matrix components present in embryonic tissue (or in stem cell niches). Likewise, their differentiation may be affected in vitro by culture with matrix components found in, or in abundance in, mature tissue. Indeed, hepatic progenitors plated onto matrix components found in association with mature parenchymal cells, such as type I collagen and certain forms of fibronectin, located in the Space of Disse near the central veins of the liver acinus, divide slowly and then cease to proliferate, which is accompanied by lineage restriction to the hepatocytic fate. The hepatic progenitors are unable to attach or survive on fibronectin, and those that do attach undergo rapid apoptosis and death. However, the hepatic progenitors give rise to descendents that require fibronectin for adhesion, survival and functions. Thus, the requirements for specific types or chemistries of extracellular matrix components are lineage dependent, that is they correlate with specific maturational stages of the cells.

The scope of the present invention should not be limited to any one matrix component or combination thereof. In keeping with the teachings herein, the present invention describes and teaches the use of any and all extracellular matrix components and their combination in the generation of substrata that can be utilized for ex vivo maintenance of cells either for expansion or for differentiation. While many of these components will be discussed below, for the sake of clarity, laminins, type IV collagens and/or type III collagens will be discussed as mere representatives of a class of extracellular matrix components that are found in or in high abundance in embryonic tissues or in stem cell niches.

Non-limiting examples of embryonic matrix components include: specific types of collagens, including Collagens Type IV (further including α1, α2, α3, α4, α5, α6) and Collagens Type III; Laminins (including, 1, γ1, β2, α3, α5); hyaluronans; forms of chondroitin sulfate proteoglycans (PGs) or their glycosaminoglycan chains; and forms of heparan sulfate-PGs or their glycosaminoglcyan chains (e.g., certain syndecans). Non-limiting examples of matrix components found in mature tissues include stable forms of collagens (e.g., type I and II), forms of fibronectin; heparan sulfate-PGs (e.g., agrin, perlecan), heparin-PGs; dermatan-PGs (e.g., cartilage-associated dermatan sulfate-PG); and elastins.

A variety of cell culture media may be appropriate with the instant invention. By adding or removing growth and/or differentiation factors from the culture medium, the rate of cell proliferation and/or differentiation may be influenced. For example, the addition of serum can slow growth of the hepatic progenitors and cause lineage restriction towards the hepatocytic fate and, in parallel, cause rapid expansion of mesenchymal cell populations (stroma and endothelia). The addition of epidermal growth factor leads to lineage restriction towards an hepatocytic fate.

Preferably, in some embodiments, the matrix components described herein are employed in combination with a serum-free medium. A serum-free media was developed previously for hepatoblasts and is described in U.S. patent application Ser. No. 09/678,953, the disclosure of which is incorporated herein in its entirety. Without being held to or bound by theory, it is presently believed that that matrix components of the present invention provide many of the survival, growth and/or proliferation signals generally provided by feeder cells. Thus, the instant invention may replace, in significant part, the need for embryonic stromal feeder cells to maintain viability and expansion potential of the hepatic progenitors.

One embodiment of the instant invention will now be described by way of non-limiting examples.

### EXAMPLES

Media & Buffers

Unless otherwise noted, serum free media was used during processing of the liver tissue and maintenance of cell cultures. This media comprises 500 ml RPMI 1640 supplemented with 0.1% bovine serum albumin, Fraction V, 10 μg/ml bovine holo-transferrin (iron saturated), 5 μg/ml insulin, 500 μl selenium, 5 ml L-glutamine, 270 mg niacinamide, 5 ml AAS antibiotic, 500 μl hydrocortisone, 1.75 μl 2-mercaptoethanol and 38 μl of a mixture of free fatty acids prepared as published in U.S. patent application Ser. No. 09/678,953. The media is sterilized and its pH adjusted to 7.4 prior to use.

“Cell Wash Buffer” comprises 500 ml RPMI 1640 supplemented with 1% bovine serum albumin, 500 μl selenium and 5 ml of AAS Antibiotic. “Enzymatic Digestion Buffer” comprises 100 ml Cell Wash Buffer supplemented with 60 mg type IV collagenase and 30 mg DNase dissolved at 37° C.

Tissue Acquisition & Preparation

Liver tissue from human fetuses between 16-22 weeks gestational age were obtained from an accredited agency, such as, for example, Advanced Biosciences Resources of Alameda, Calif. Preferably, tissues were received within 18 hours of isolation and arrived as multiple sections of liver tissue or, on occasion, as reasonably intact liver. In general, overall tissue volumes ranged between about 4 ml and about 12 ml and contained large quantities of red blood cells (RBCs).

The livers were mechanically dissociated, and the tissue was partially digested with the enzymatic digestion buffer yielding clumps of parenchymal cells. These clumps were subjected to washing and low speed centrifugation to substantially eliminate free floating hemopoietic cells yet retain hepatic parenchyma. The dissociated livers were then segmented into 3 ml aliquots to which 25 mls of Enzymatic Digestion Buffer was added. After 30 minutes of moderate agitation at 32° C., the supernatant was removed and stored at 4° C. Any residual unfragmented pellets were re-digested with fresh Enzymatic Digestion Buffer for an additional 30 minutes. After completion of the enzymatic digestion of the tissue fragments, the cell suspensions were centrifuged at 250 revolutionary centrifugal force (RCF); the supernatants removed; and the pellets resuspended in an equivalent amount of Cell Wash Buffer.

Isolation Techniques

Liver cell suspensions from fetal livers are replete with hemopoietic cells, especially erythroid cells. In fact, original cell suspensions of human fetal livers consist, on average, of only 6-9% parenchymal cells with the remainder being various non-parenchymal cells, particularly erythroid cells. As routine methods for elimination of erythroid cells, such as use of a lysing buffer, may be toxic for the hepatic progenitors other methods may be preferable. For example, the erythroid cells may be separated from the parenchymal cells by repeated slow speed centrifugations using methods published previously (Lilja et al., 1997; Lilja et al., 1998).

An alternate and more efficient method, complement-mediated cytotoxicity, can be utilized to minimize the loss of candidate stem cells. After collagenase digestion, anti-human red blood cell (RBC) antibodies can be incubated with the cell suspension (1:5000 dilution) for 15 min at 37° C. To puncture and lyse antibody-marked erythrocytes, complement (e.g., LowTox Guinea Pig complement) is added (1:3000 dilution) for a 10 min incubation at 37° C. Cell supernatant will become pinkish from hemoglobin released from erythrocytes. Suspensions purged of hemopoietic cells consist of at least 80-90% parenchymal cells.

The resulting suspension, from which the hemopoietic cells have been purged, is then subjected to a second round of enzymatic digestion in fresh collagenase solution for 30 min to minimize cell clumping, followed by sieving through a 75 μm nylon sieve. Estimated cell viability by trypan blue exclusion was routinely higher than 95%. After filtration, most hepatoblasts were visualized as clumped cells containing 4 to 8 cells per aggregate. Together these techniques help to generate cell suspensions substantially free of red blood cells yet enriched parenchymal liver cells.

Further enrichment protocols include Ficoll Fractionation for isolation of hepatoblasts. Briefly, cells are suspended in 10 ml of basal medium without phenol red and overlayed onto an equal volume of Ficoll-Paque (Amersham Pharmacia) in a 50 ml centrifuge tube. Cells are subsequently centrifuged at 1000×g for 25 minutes. Both the interface and the pelleted cells are collected. Ficoll fractionation yields pellets that are generally more than 80% parenchymal cells, essentially all of which are hepatoblasts, and an interface of 13-14% of the original cell population with diverse antigenic profiles indicating parenchymal, hemopoietic and endothelial cells. The Ficoll interface cells yield colonies of ductal plate cells at 0.1%, a 10-fold increase over that from the original cell suspension. While results with Ficoll fractionation may be consistent for isolation of hepatoblasts, they tend to be more variable from prep to prep in the isolation of the stem cells.

Immunoselection is another technique for enrichment and/or isolation of hepatic stem cells (ductal plate cells) or other subpopulations. Preferable immunoselection protocols are those which employ antigens with intense expression on cells (e.g., EpCAM found on all hepatic progenitors) and others on one subpopulation of hepatic progenitor (e.g., NCAM on ductal plate cells). Immunoselection can be by any of the diverse methods for these procedures and can be cytometers, panning, or magnetic beads.

Magnetic Immunoselection comprises the isolation of cells expressing, for example, EpCAM from human liver cell suspensions using monoclonal antibody HEA125 coupled to magnetic microbeads, and an autoMACS™ or CliniMACS® magnetic column separation system from Miltenyi Biotec (Bergisch Gladbach, Germany), following the manufacturer's recommended protocols. Similar methods were used for immunoselection of NCAM, CD146, KDR (VEGFr), and CD133/1 cells.

Assays

For all assays performed, 6-well petri dishes were utilized and 0.8×10⁶ parenchymal cells were seeded per well. For the first 10 hours after initial seeding, the medium was supplemented with 10% fetal bovine serum. It is believed that supplementation with serum during initial plating facilitates inactivation of the enzymes used in tissue digestion and assists initial attachment. Thereafter, only serum-free medium was used. Generally, the medium was changed at 24-hour intervals, and in some cases, at 3-day intervals. Unless otherwise noted, experimental values were normalized to nutrient media volumes and cell numbers.

Urea

Assays based on direct interaction of urea with diacetyl monoxime were conducted to determine the urea concentration of collected media samples. Standards and reagents were purchased as diagnostic kits from Sigma. The kits were modified for use with a 96-well microplate. For this modification, diluted concentration standards were initially prepared. Using serial dilutions, the standard concentrations were decreased to yield a range of standards from 0-30.76 mg/dL. Then a combined reagent (“Reagentcombined”) is made by mixing the blood-urea-nitrogen (bun) acid reagent and bun color reagent into a 1.3 to 1.0 ratio.

The assay protocol consisted of first loading 9 ml of the appropriate sample (e.g. blank, standard, or sample) in each microplate well followed by 100 ml of Reagentcombined. The loaded microplate was then warmed at 50° C. for approximately 25 minutes or until distinct standard color changes were visible within the concentration standards. Next, the base of the microplate was ice-chilled for 3 minutes. Immediately after cooling, the optical density variances were measured at 535 nm using a cytofluor multi-well reader.

Ammonia—NH₃

Colorimetric assays based on reacting NH₃ with bromophenol blue (ammonia indicator) were analyzed with the VITROS DT60 II chemistry system (Ortho-Clinical Diagnostics, Rochester N.Y.). The automated process requires a 10 μl sample, a 5 minute incubation time, a 37° C. environmental chamber, and a 605 nm absorbance measurement cube. Once inside the chemical analyzer, the slide is labeled for optical density variations and plotted against precalibrated standards.

Glucose

The VITROS DT60 II chemistry system uses a dual reaction sequence. Initially, the oxidation of sample glucose is catalyzed by glucose oxidase to form H₂O₂. This reaction is followed by an oxidative coupling catalyzed by peroxidase in the presence of dye precursors, in which the intensity of dye is measured by reflected light. Individual assays consist of first loading 10 μl of the appropriate sample (e.g., blank, standard, or sample) onto each chemistry slide. Once inside the chemical analyzer, the slide is labeled for optical density variations and plotted against precalibrated standards.

Immunostaining

Immunostaining of cells was done after culture fixation using a 50/50 mixture of acetone and methanol for 2 minutes, washed again with 1×PBS, and blocked with 10% goat serum for 45 minutes. Then a primary human antibody, conjugated with a fluorescent probe, was added for 1 to 8 hours at room temperature. When unconjugated primary antibody was used, the cells were stained with a secondary antibody conjugated with a fluoroprobe.

Proliferation

Proliferation of hepatic progenitors was assessed macroscopically by imaging size changes of colony growth using phase microscopy with 4×, 10×, and 20× magnifiers; the low magnification objectives allowed observation of entire colonies. Growth curves were obtained by repetitive colony imaging, and the micrographs normalized against known dimensions precalibrated on MetaMorph Image Software for statistical comparative analysis.

Preparation of Tissue Culture Matrices

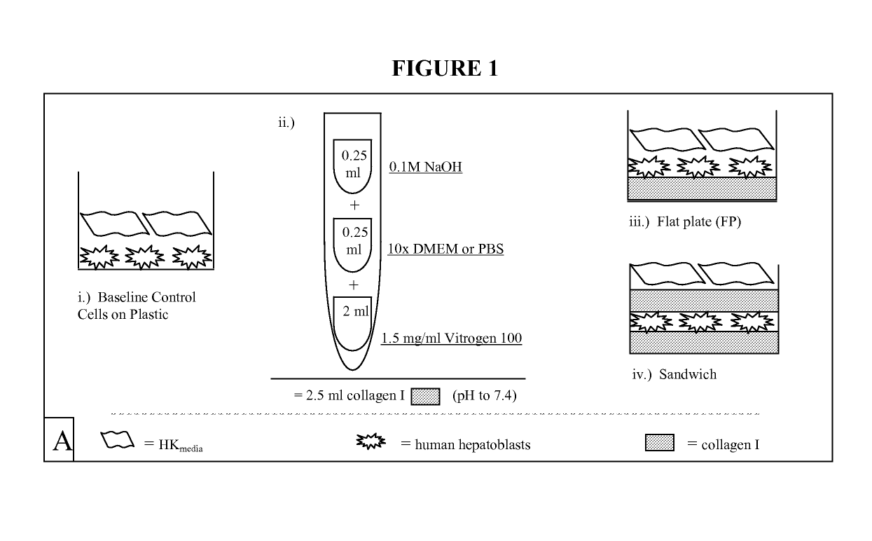

Control studies included seeding fetal human liver cells directly onto tissue culture plastic (TCP) (FIG. 1A). Fibronectin plates were prepared at three different concentrations (0.5, 1.0, or 2 μg/cm²) and adjusted to pH 7.5. Collagen type-I plates were prepared at concentrations of 1 to 1.5 mg/ml. For this preparation, high density Vitrogen 100 (Cohesion Technologies, Palo Alto, Calif.) was modified into liquid collagen type-I by adding specific ratios of 10×DMEM and 0.1M NaOH. More specifically, FIG. 1B shows one embodiment in which 0.25 ml 0.1M NaOH, 0.25 ml 10×DMEM or PBS, 1.5 mg/ml Vitrogen 100 are combined and gently mixed at 4° C. to generate a homogeneous solution. During this process it may be desirable to avoid introducing air bubbles within the newly formed collagen I suspensions as air gaps can destabilize the collagen.

The collagen I suspensions were utilized to precoat petri well surfaces for both Flat Plate (FP) and “sandwich” designs as shown in FIGS. 1C and 1D, respectively. FPs were prepared with 0.4 ml of collagen I suspension into each well of a six well plate. The collagen I suspensions were allowed to gel at 37° C. and 5% CO₂ for 1 hour. After gelation, the plates/wells were ready to receive the hepatic progenitors.

Sandwich plates may be prepared in an identical manner to that of FPs. However, in order to “sandwich” the cells, a second layer of collagen suspension is poured onto an FP with attached cells thereon. As with the FPs, the sandwich plates were incubator-gelled at 37° C. and 5% CO₂ for 1 hour to solidify the new top collagen layer. After gelation, 0.5 ml of serum free media was added for nutrient support.

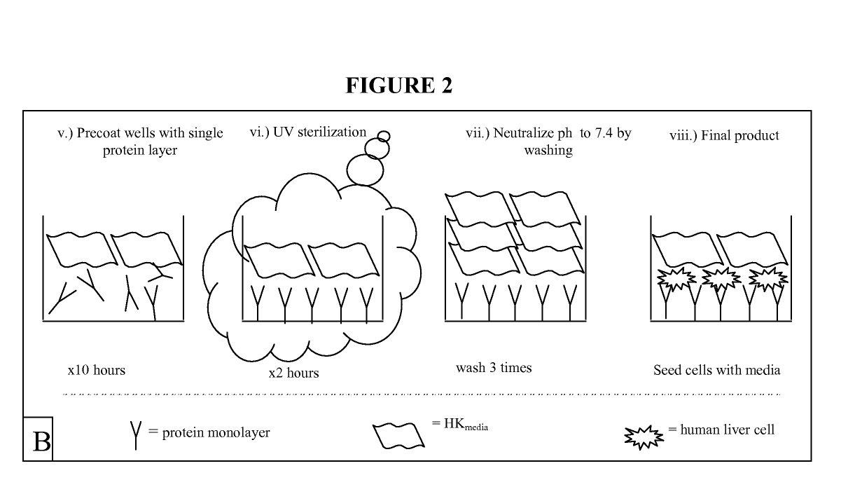

Laminin coated plates were prepared at two different concentrations (0.52 or 1.0 ug/cm²) and adjusted to pH 7.5. Collagen coatings were prepared on dishes utilizing 1 of 5 different protein concentrations (2.1, 4.2, 6.3, 8.3 or 10.4 μg/cm²). Following coating, the matrix was allowed to attach for 10 hours at 37° C. and 5% CO₂. The coating process is illustrated in FIG. 2, where random matrix molecules were heterogeneously distributed within acetic buffers and inside the dishes. Within about 10 hours, the matrix molecules stabilized and attached to the well surface in single homogeneous arrays. The plates were subsequently UV sterilized for 2 hours and rinsed with 1×PBS to neutralize the acidic pH. Collagen III plates were similarly prepared with pH 3 acetic acid and Collagen IV plates with 0.5 M acetic acid.

Where plated in combination, type III collagen and laminin were co-plated on TCP surfaces such that their individual concentrations were 6.25 μg/cm² and 0.52 μg/cm² respectively. Type IV collagen and laminin were co-plated on TCP surfaces such that their individual concentrations were 4.2 μg/cm² and 1.0 μg/cm², respectively.

Identification of the Matrix Components in the Liver's Stem Cell Compartment

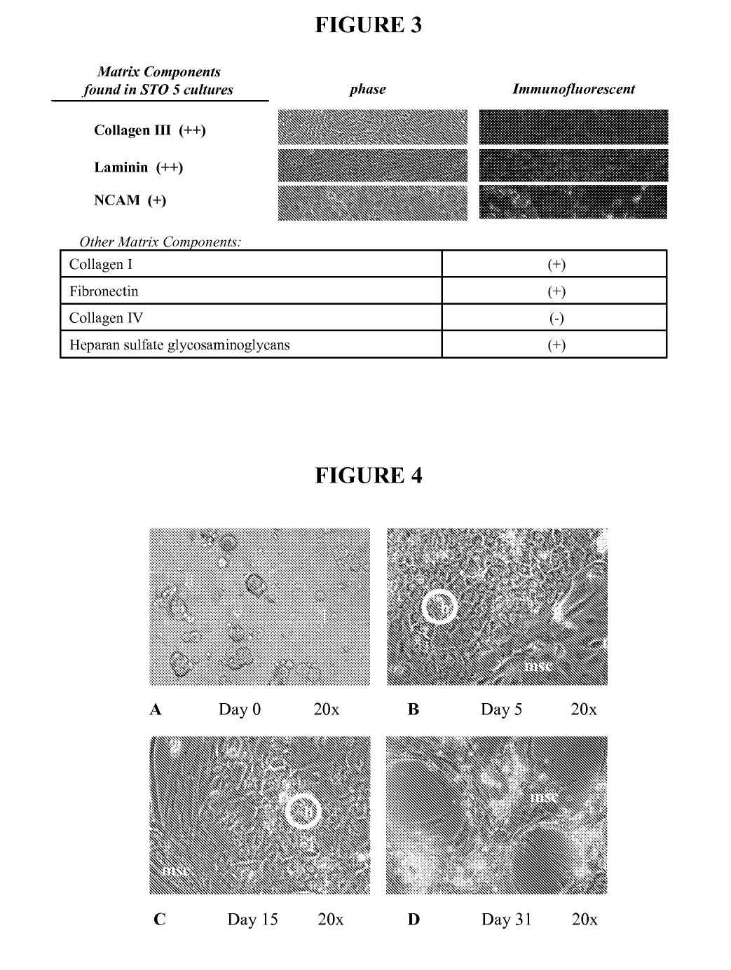

The matrix components in the stem cell compartment was identified in vivo by immunohistochemistry on fetal livers sections and in vitro by assays for matrix components produced by embryonic mesenchymal feeder cells. Angioblasts, which are the native mesenchymal partner for hepatic stem cells, and murine embryonic stromal feeders (e.g., STO cells) were used as exemplary cells for these experiments. As shown in Table I, the study determined that the feeder cells (e.g., angioblasts and the STO feeders) produce laminin, type III collagen and type IV collagen, hyaluronans and heparan sulfate proteoglycan. Of note, hepatic stem cells have receptors for both hyaluronans (FIG. 3) and the collagens identified.

| | | In Vitro Assays (in | | |
|---|---|---|---|---|
| | | presence of hepatic | | In Vivo |
| | Location | stem cells) | | Assays |
| | Liver | STO | Angio- | Ductal |
| Matrix Component | Acinus | Feeders | blasts | Plates |
| Laminin | Periportal | ++ | + | + |
| Fibronectin | Pericentral | + | − | − |
| Type I Collagen | Pericentral | + | − | − |
| Type III Collagen | Periportal | ++ | ++ | ++ |
| Type IV Collagen | Periportal | ++ | ++ | ++ |
| Hyaluronan | Periportal | ++ | ++ | ++ |
| Heparan Sulfate (HS) | Periportal | + | + | + |
| Proteoglycan | | | | |
| Heparin Proteoglycan | Pericentral | − | unknown | − |
| Chondroitin Sulfate (CS) | Periportal | + | unknown | unknown |
| Proteoglycan | | | | |
| Dermatan Sulfate (DS) | Pericentral | not done | unknown | unknown |
| Proteoglycan | | | | |

A more detailed comparative survey is presented in Table II.

| | | Stem Cell Niche: Ductal Plate | | | Mature Liver: Space of Disse | |
|---|---|---|---|---|---|---|
| | | | Type III | Type IV | Type I | |
| Marker/Function | Characteristic | Laminin | Collagen | Collagen | collagen | Fibronectin |
| Attachment | | + | + | + | + | Limited |
| Survival | | + | + | + | + | Rapid Apoptosis |
| Spheroid vs Monolayer formation | | Spheroid | Monolayer | Monolayer | Monolayer | Spheroid |
| | | Formation | | | (3D) | |
| Colony Formation (somatic cells) vs. Clonogenic | | Clonogenic | Clonogenic | Clonogenic | Colony | No growth; |
| Expansion (stem cells) | | expansion | Expansion | Expansion | formation | apoptosis |
| Division Rate (doubling time in hours) | | not done | <24 hours | <36 hours | >3-5 days | — |
| | | | | | or slower | |
| Albumin | Hepatocytic Marker | + | + | + | ++ | + |
| Cytokeratin 19 (CK19) | Biliary Marker | + | + | + | − | − |
| Alpha-fetoprotein (AFP) | Hepatoblasts & committed | − | − | + | ++ | − |
| | hepatocytic progenitors | | | | | |
| E-Cadherin | Cell Surface Marker | + | + | + | +/− | + |
| | of Epithelia | | | | | |
| Ep CAM | Hepatic stem cells, | ++ | ++ | ++ | + | + |
| | hepatoblasts, committed | | | | | |
| | progenitors | | | | | |
| N-CAM | Hepatic stem cells | + | + | + | − | − |
| Cytokeratins 8 & 18 | Markers of Epithelia | + | + | + | + | + |
| (CK 8/18) | | | | | | |
| Glucose Production | Function of periportal | not done | − | + | + | − |
| | hepatocytes | | | | | |
| Urea production | Function of mature, | not done | − | ? | + | Did not survive |
| | pericentral hepatocytes | | | | | long enough to |
| | | | | | | assay |

The survey in Table II describes human hepatic stem cell responses, including cell attachment, cell survival, geometric culture formations, unique colony formations, division rates, immunohistochemistry responses, along with functional glucose and urea productions. For attachment, human hepatic stem cells established cell matrix interactions when cultured on either laminin or type III or IV collagens or the adult substratum type I collagen. The only substratum surveyed on which there was minimal attachment was on fibronectin. Furthermore, cell survival of greater than 10 days is evident on all matrix substrates except fibronectin, which promoted rapid apoptosis for the few cells that attached. This is noteworthy, since mature parenchymal cells require fibronectin for attachment, survival and functioning.

Next, the geometric formations of the cultures were observed. Laminin and fibronectin induced 3D spheroid aggregates while other substrates induced monolayers of the cells. Furthermore, clonogenic expansion was observed for cells plated on laminin, type III collagen, and type IV collagen; while either colony formation or no-growth were observed for type I collagen and fibronectin respectfully. Furthermore, type III collagen induced division rates of less than about 24 hours, whereas cells seeded onto type I collagen slowed and then went into growth arrest and then maintained the cells as viable and functional.

Immunohistochemistry responses are also compared for albumin (ALB), Cytokeratin19 (CK19), α-Fetoprotein (AFP), E-Cadherin (E-CAD), Epithelial Cell Adhesion Molecule (EpCAM), neural cell adhesion molecule (NCAM), and Cytokeratins 8 and 18 (CK8 and 18). The most dominant and positive response is displayed as NCAM+, EpCAM (++), ALB (+) and CK8 and CK18 (+). Interestingly, CK19 was strongly expressed by stem cells cultured on laminin, type III collagen, and type IV collagen and negative for those plated onto type I collagen and fibronectin. This finding suggests early bipotent activities are lineage restricted by matrix components in abundant in mature tissue. Moreover, the high AFP and ALB activities for cells on collagen I indicate parenchymal cells undergoing lineage restrictions toward committed hepatocytic progenitors, a finding corroborated by data indicating strong expression of glucose and urea production.

Turning first to hepatic progenitors plated directly onto the TCP surface morphological characteristics of human hepatoblasts and hepatic stem cells and associated mesenchymal cell partners were followed over a 31-day time-lapse investigation. On day 0, liver cells were homogeneously distributed onto TCP surfaces. The initial hepatoblast (h) populations were organized as spheroid clumps containing 3-8 hepatoblasts ( [symbol] ) but were also found as single cells (↑), as illustrated in the 20× micrograph of FIG. 4A. In this figure, the hepatoblasts were identified by an average diameter of about 10 μm to about 12 μm, whereas the smaller cells with a diameter of less than about 10 μm were deemed either mesenchymal cells, residual RBCs or hepatic stem cells. Cell cultures were non-confluent with multiple heterogeneous spheroid aggregates attached on the surface.

By the 5th day of culturing, the residual RBCs had detached from the TCP surface and died. The remaining cell populations, largely hepatoblasts and various mesenchymal cells (mcs), were stabilized into their respective morphologies as shown in FIG. 4B. This stabilization promoted hepatoblast (h) culture confluency as evidenced in hepatoblast populations that were flattened, spread, and had initiated cell-cell contact. Furthermore, hepatoblasts retained good cellular distinctions with sharp membrane contours and smooth cytoplasmic features. Additionally, limited co-culture interactions were present with few and discrete shared boundaries between hepatoblast and mesenchymal cell types.

By day 15 (FIG. 4C), hepatoblasts had “grainy” membranes and cytoplasmic features consistent with deteriorating cells. Additionally, the ratio between hepatoblast and mesenchymal cells had equalized, suggesting non-parenchymal cells were occupying more surface area with the hepatoblasts eliminated. This finding paralleled the classic phenomenon of “fibroblast overgrowth,” even in the absence of serum supplementation of the medium. These changes indicated TCP culture conditions were conducive for mesenchymal cell expansion and not preferable for hepatoblast proliferation and survival. Indeed, on day 31, hepatoblasts had either died or reorganized into scaffolding as mesenchymal cells overwhelmed the culture surface, as shown in the 20× micrograph in FIG. 4D.

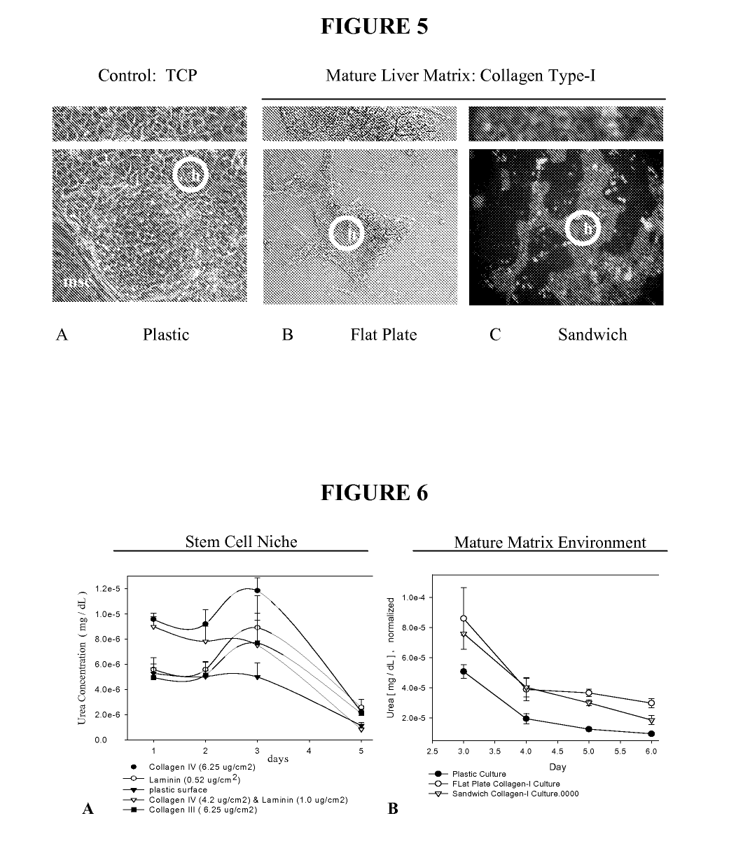

A comparison was next made of human hepatic progenitors when cultured on TCP alone versus FP or sandwich collagen type-I gels. Day 10 cultures are shown in FIG. 5. For the culture control of “cells on TCP” shown in FIG. 4A, hepatoblasts (h) displayed granular cytoplasmic features with indistinct cell borders. Additionally, mesenchymal cells (mcs), largely endothelia and stoma, surrounded the hepatoblast colonies. The appearance of the hepatoblasts (h) on plastic was strikingly different from that when seeded on collagen type-I (FP) for the same time in culture (FIG. 5B). The hepatoblasts showed definitive cell borders and agranular cell cytoplasm—characteristic of stable hepatoblasts. Additionally, mesenchymal cells showed limited proliferation but also retained prominent cell border characteristics.

Hepatoblasts cultured using a sandwich design illustrated in FIG. 4C demonstrated established cell-cell contacts, definitive cell borders, and dual expression of hepatocytic and biliary functions as evident by immunostaining for albumin (red) and CK19 (green) expression. The results were indicative of stabilization of bipotency in this progenitor population.

Urea is produced uniquely by mature liver cells. Thus, its expression can be indicative of tissue-specific gene expression and a significant extent of differentiation. Accordingly, in order to study the extent of differentiation of the immature hepatic cells as a function of the in vitro matrix proteins, the concentration of urea in the media was determined after several hours in culture.

FIG. 6 shows hepatoblasts cultured on extracellular matrices which are dominant in embryonic and fetal liver tissues. A partial listing of these in vivo matrices includes collagen III (▪), collagen IV (●), laminin (∘), and a mixture of collagen IV-laminin [symbol] which are analyzed and compared against TCP controls (▾). For urea analysis, hepatoblasts were monitored for days 1-5 and their normalized urea results are displayed on the vertical axis.

Overall, the control cells on TCP (▾) displayed inferior activity throughout the investigation—having maximum and minimum urea magnitudes of 5.5×10⁻⁶ and 1.5×10⁻⁶ mg/dL, respectively. More specifically, immediate distinctions were observed on day 1 when hepatoblasts seeded on either ‘collagen IV’ or the ‘collagen IV and laminin mixture’ reached urea levels of 9.5×10⁻⁶ mg/dL; all other cultures assayed had urea levels near 5.3×10⁻⁶ mg/dL—or 56% deviations between highly active and sedentary hepatoblasts. Few changes were noted between the day 1 and day 2 results, except hepatoblasts seeded on ‘collagen IV and laminin’ showed evidence of slight declines in urea function. By day 3, however, the collagen IV (●) cultures revealed the best urea function activity reaching 1.2×10⁻⁵ mg/dL, whereas the other cultures expressed 33% lower urea levels. By day 5, all urea levels merged to minimal urea expression levels of 2×10⁻⁶ mg/dL suggesting that the ammonia was depleted from the culture media since urea expression requires ammonia factors.

Urea production is expressed as ‘per cell’ concentrations of urea (FIG. 6B). These plots represent the concentration of urea over a 24-hour period and are labeled such that circular ‘solid black (•)’ data points are hepatoblasts on TCP (control), circular ‘solid white (∘)’ data points are hepatoblasts from cultures seeded on of collagen type-I (flat plate, FP), and triangular [symbol] ‘gray’ data points are hepatoblasts cultured between collagen type-I layers (sandwich design), respectively. In this way, the time-lapse evaluations were analyzed for culture days 3 thru 6.

As illustrated for each system, the urea concentration levels were high at day 3 but continually decreased throughout the investigation. Also noted were lower urea levels in the control throughout the entire study. Hepatoblasts cultured in FP and sandwich configurations had similar activities of 8.0×10⁻⁵ and 5.0×10⁻⁶ mg/dL for days 3 and 4, respectively. These values correspond to ˜80% activity improvements when compared to control cultures on culture plastic. As well, the Flat Plate system revealed higher hepatoblast activity on days 5 & 6. As displayed, the hepatoblast FP activity exhibited 8% better activity than Sandwich and ˜115% better activity than TCP analyzed cultures.

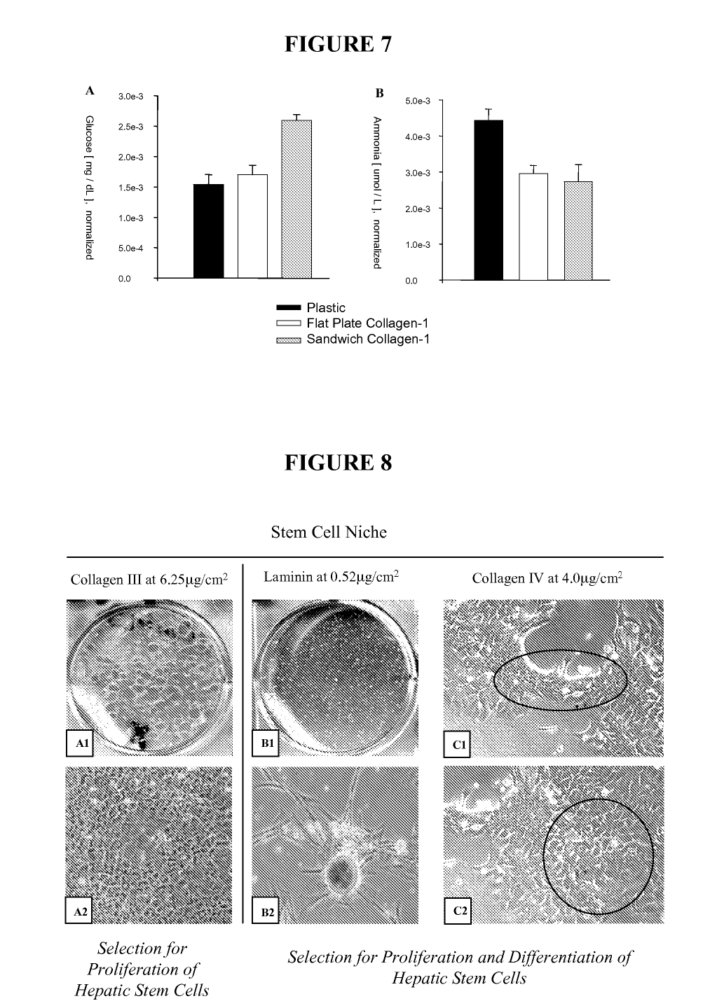

Two additional assays were used to compare functional activity for cells seeded on the various matrices. FIG. 7 depicts glucose and ammonia production, respectively, on day 3, which was shown to be the time of highest daily urea activity (FIG. 6). The glucose comparison in FIG. 7A shows TCP culture control levels at 1.5×10⁻³ mg/dL—labeled in solid black (▮), collagen type-I FP culture levels at 1.63×10⁻³ mg/dL labeled in solid white (∥), and sandwich collagen type-I culture levels at 2.6×10⁻³ mg/dL—labeled in solid gray. Comparisons between sandwich and TCP or FP cultures showed sandwich cultures with ˜64% higher glucose levels than other cell-system responses.

FIG. 7B displays ammonia accumulation in TCP cultures at 4.5×10⁻³ mmol/L, in FP systems at 2.8×10⁻³ mmol/L and sandwich collagen systems at 2.6×10⁻³ mmol/L concentration magnitudes. The FP and sandwich cultures had decreased ammonia levels and controls displayed ammonia levels at ˜60% greater magnitudes.

Hepatoblasts demonstrated morphological changes dictated by the chemistry of their substratum, as indicated in FIG. 8. FIGS. 8A1, 8B1 and 8C1 show low magnification images and overall surface characteristics of day 10 cultures. FIGS. 8A2, 8B2 and 8C2 show detailed morphology distinctiveness with higher image magnifications of identical cultures. In FIG. 8A1, the petri surface was precoated with 6.25 mg/cm² collagen III prior to seeding fetal liver cells. At day 10, high quantities of interconnected tissue masses and cohesively gelled substratum was established. Additionally, this culture showed recently developed, but randomly dispersed, acellular and circular subdivisions. For more detailed image analysis, micrograph FIG. 8A2 shows a magnified segment of FIG. 8A1. This enhancement shows human hepatoblast networks in closely grouped patterns.

To contrast this cell-environmental effect, the hepatoblast stocks were cultured onto 0.52 mg/cm² laminin-coated petri surfaces, as shown in FIGS. 8B1 and 8B2. In FIG. 8B1, large quantities of segmented “white specks” were dispersed throughout the seeding surface. These groups were validated in FIG. 8B2 as tightly compact spheroid aggregates with elongated spheroid-to-surface contacts. As the spheroids grew and shifting media forces caused large spheroids to sway, many of the surface attachment bonds broke and the cells are washed from the cultures. However, the spheroids could be transplanted onto collagen I and collagen III substrata to again induce cell attachment and future spreading.

For a third contrast of cell-matrix effects, the same fetal liver cells are cultured onto pre-coated collagen III-laminin petri surfaces at concentrations of 6.25 and 0.52 mg/cm² respectively. As displayed in FIG. 8C1, the overall culture effect showed high quantities of interconnected tissue masses using white backgrounds. Heterogeneous acellular areas free of tissue and matrix components were also evidenced. Likewise, FIG. 8C2 shows co-cultured hepatoblasts and non-parenchymal that were active and stable. The majority of cells were hepatoblasts with “cobblestone” culture arrangements. Additionally, some border interactions are between parenchymal and non-parenchymal cell partners were observed.

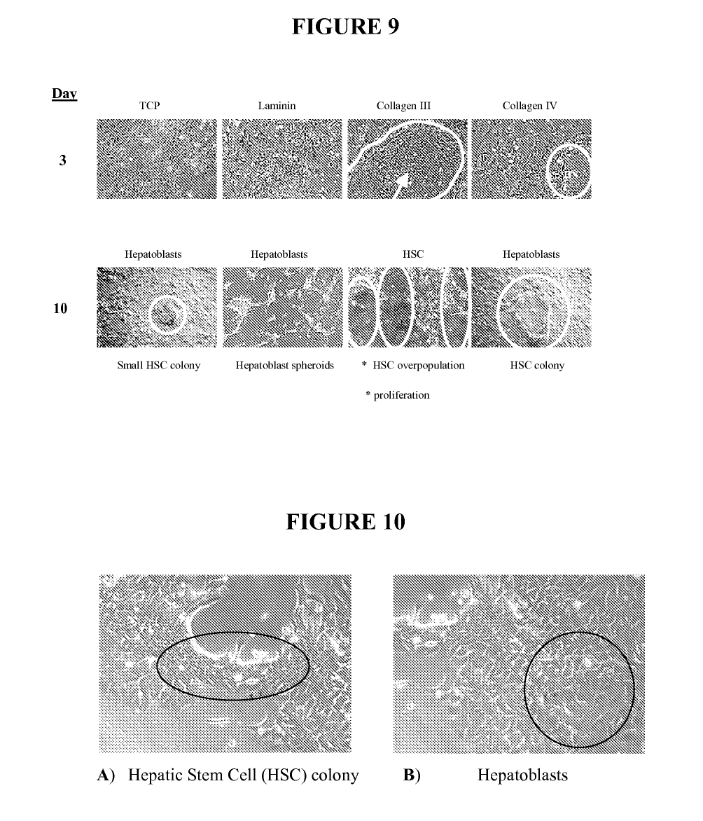

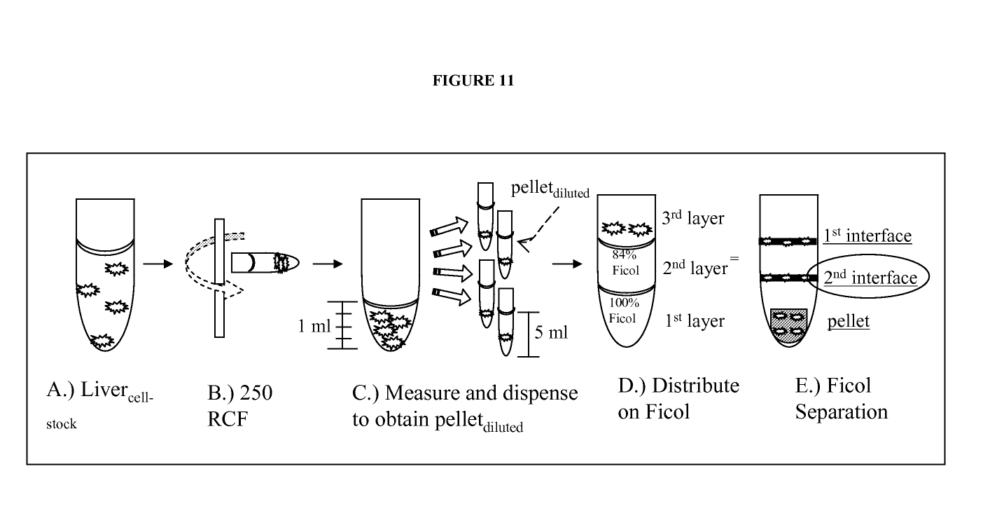

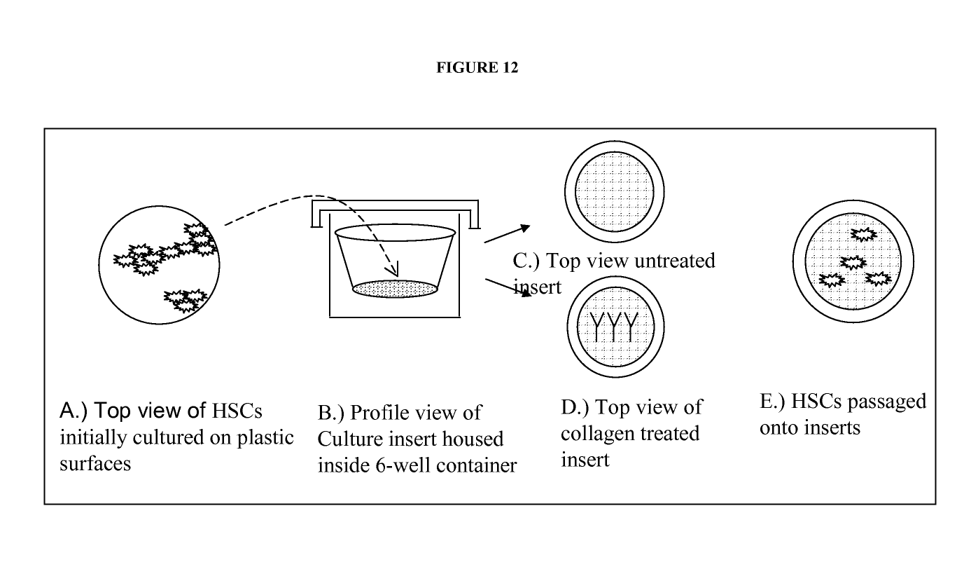

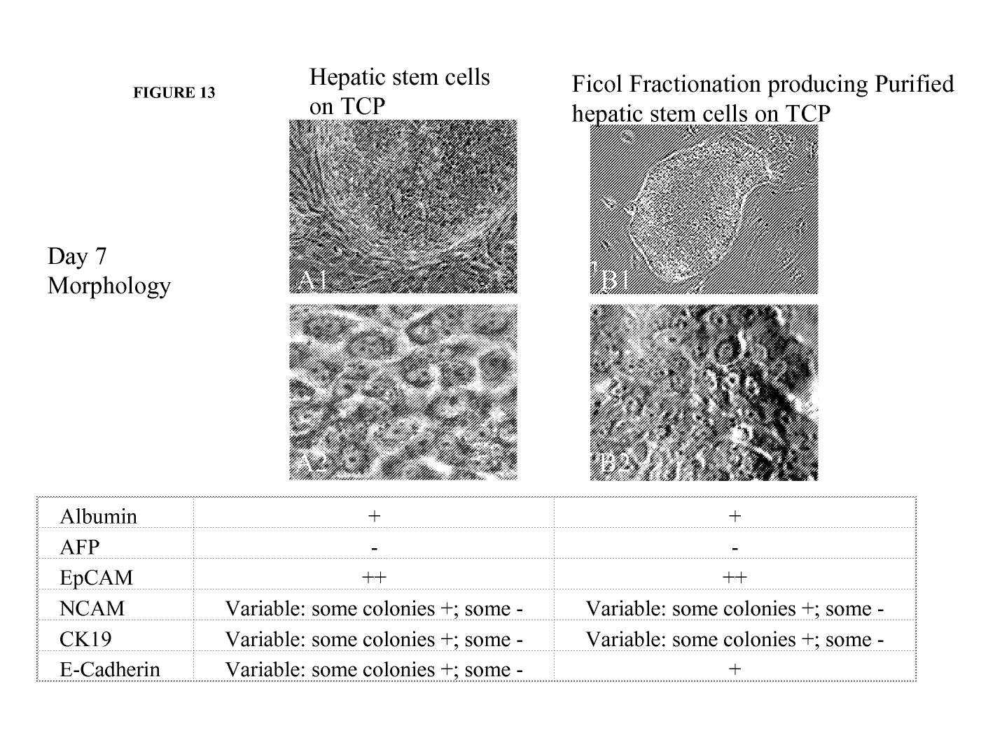

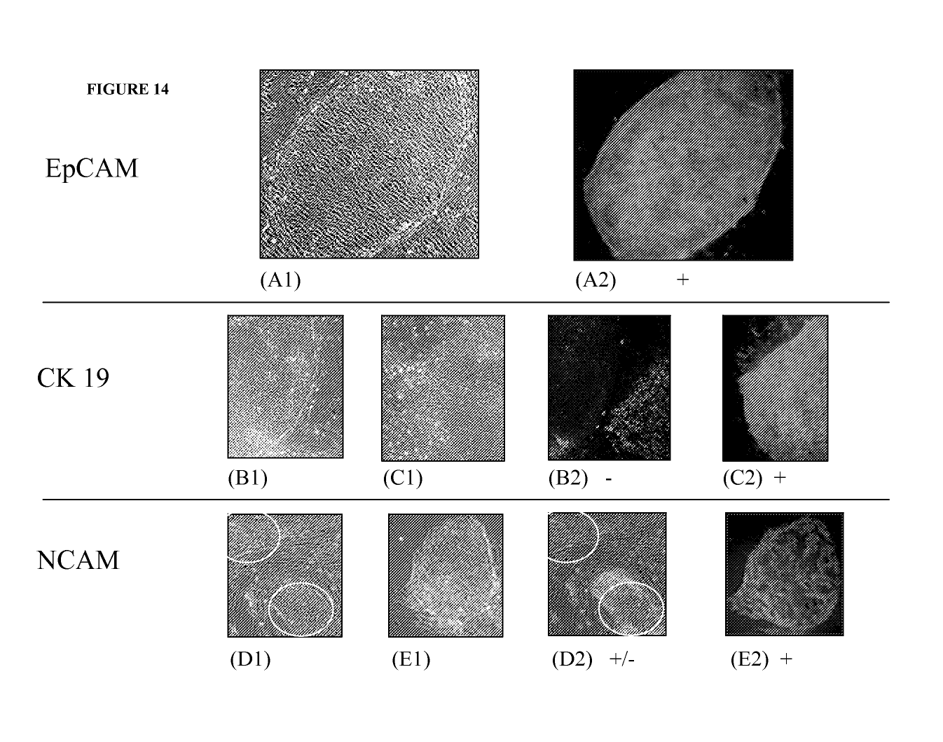

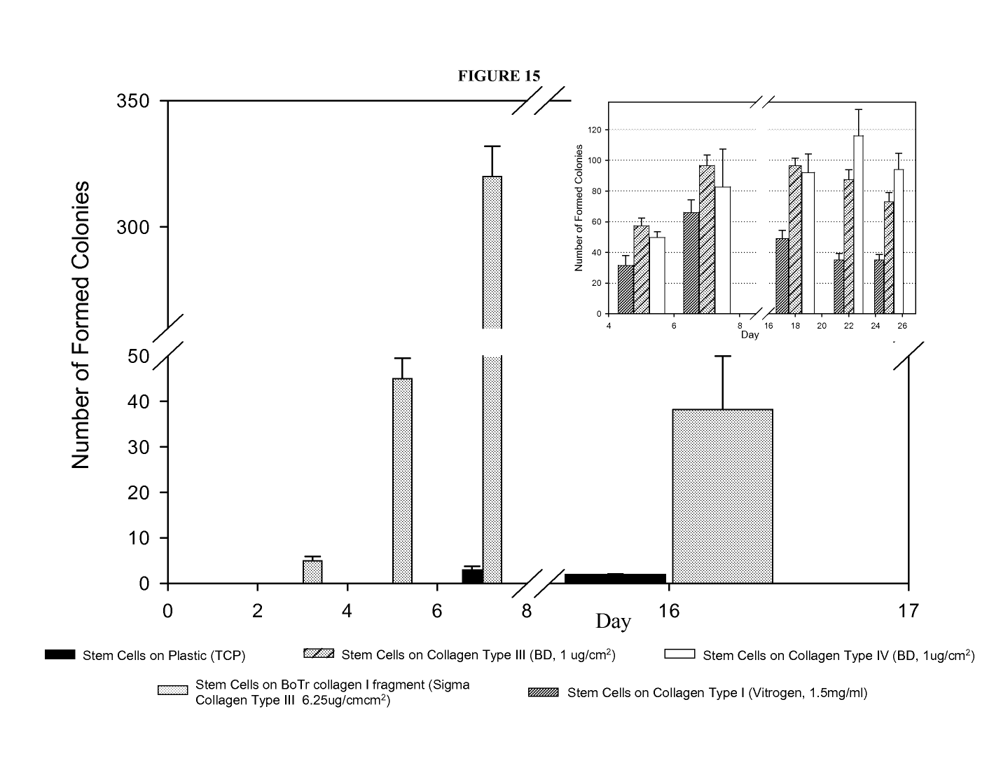

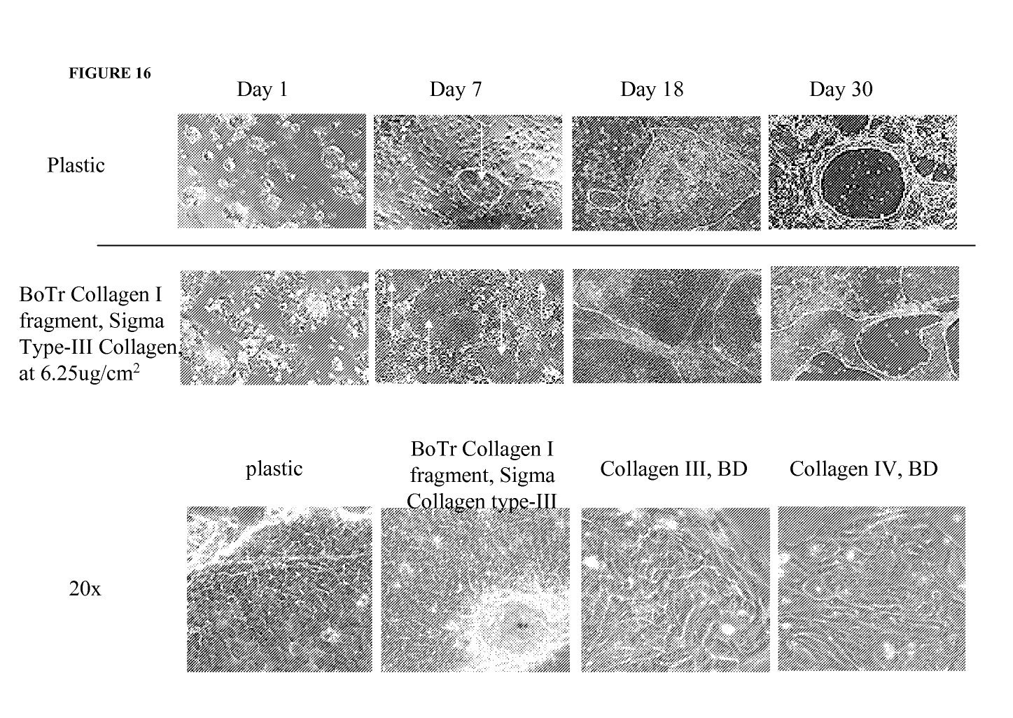

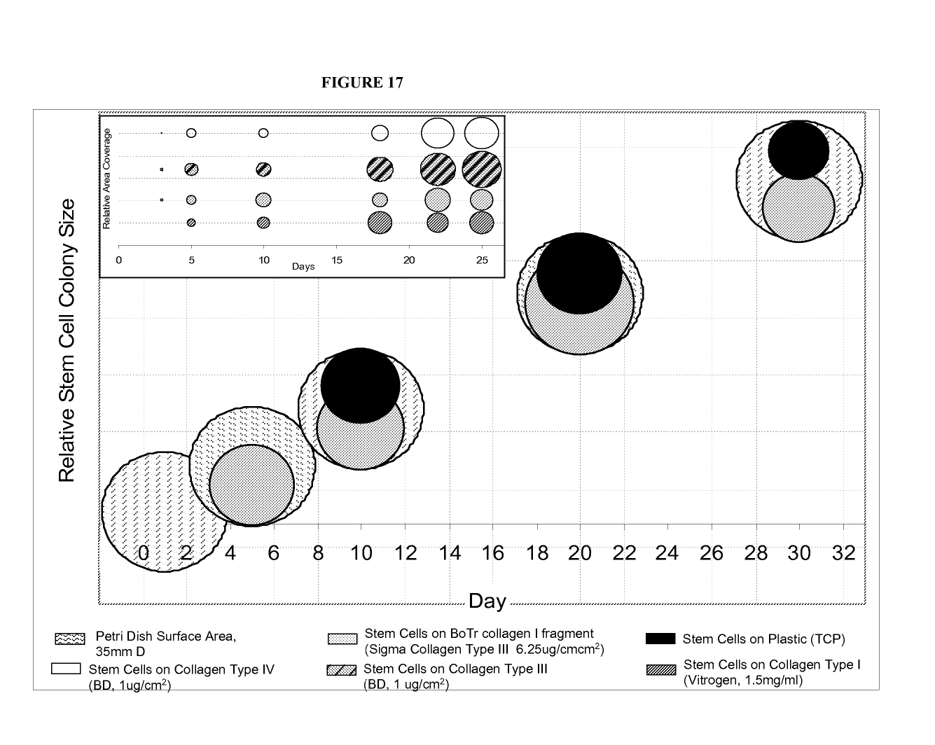

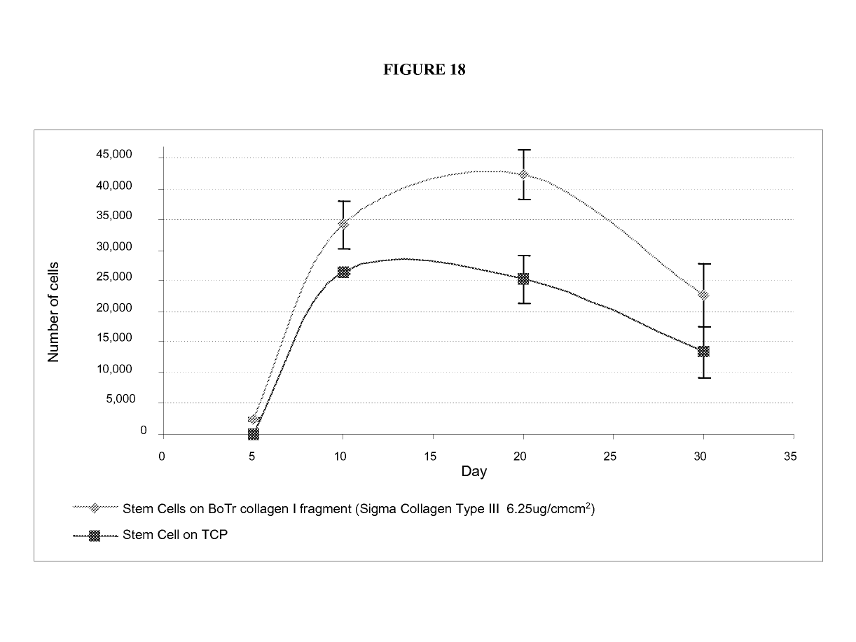

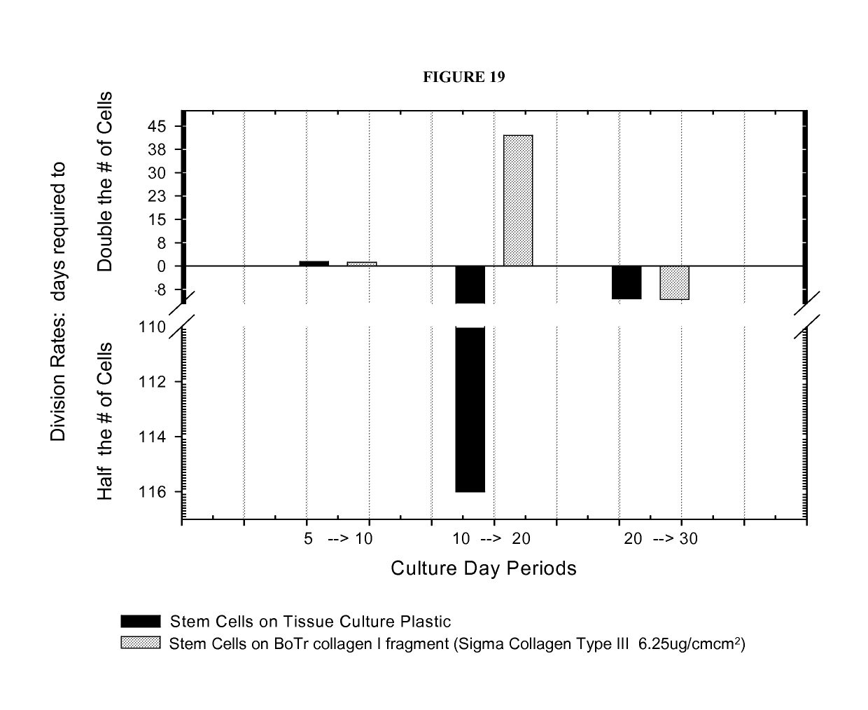

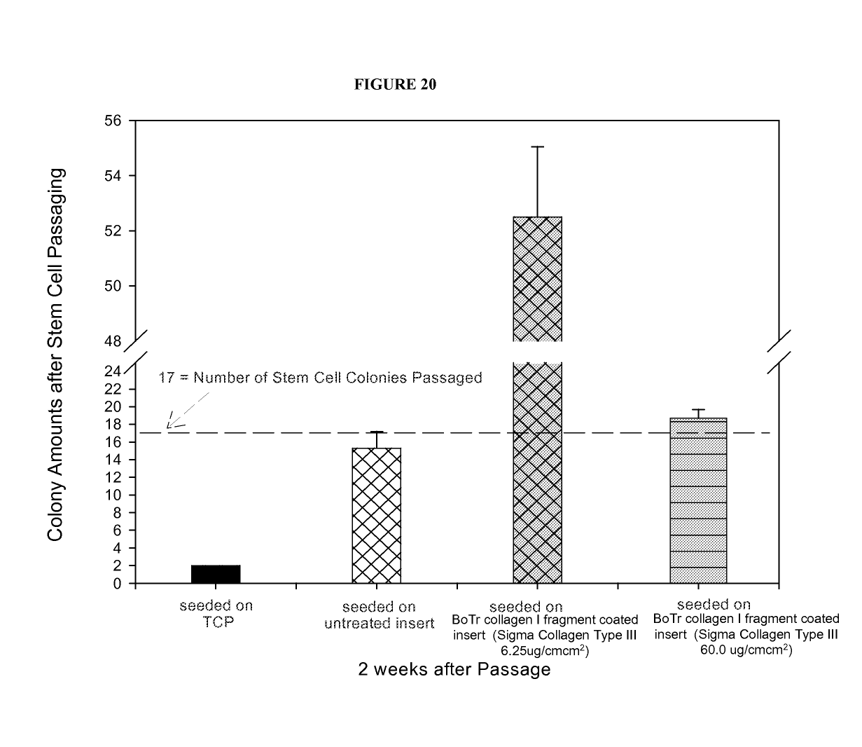

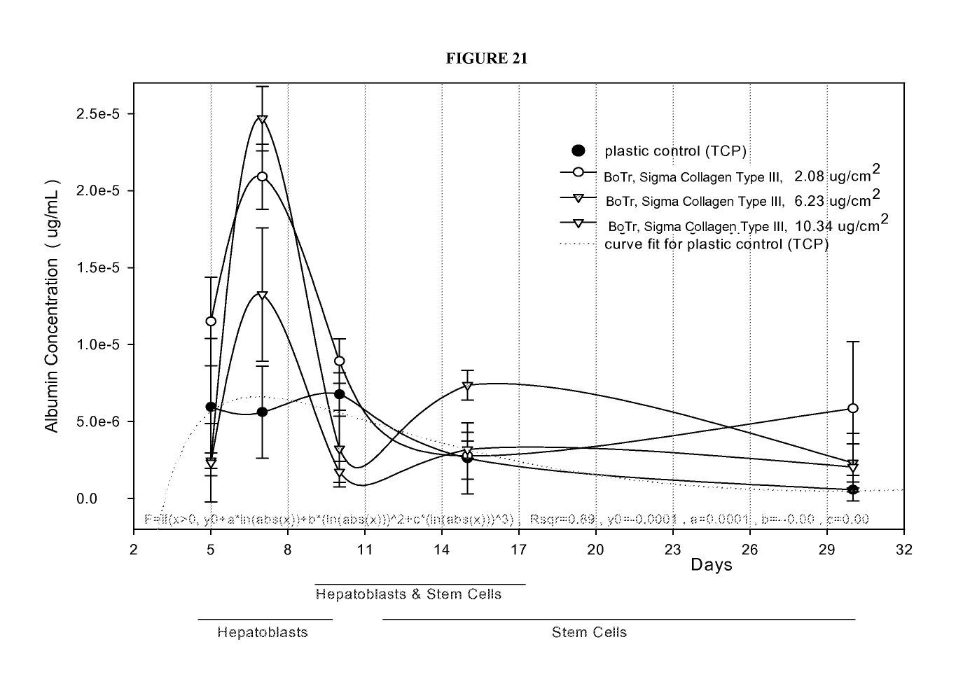

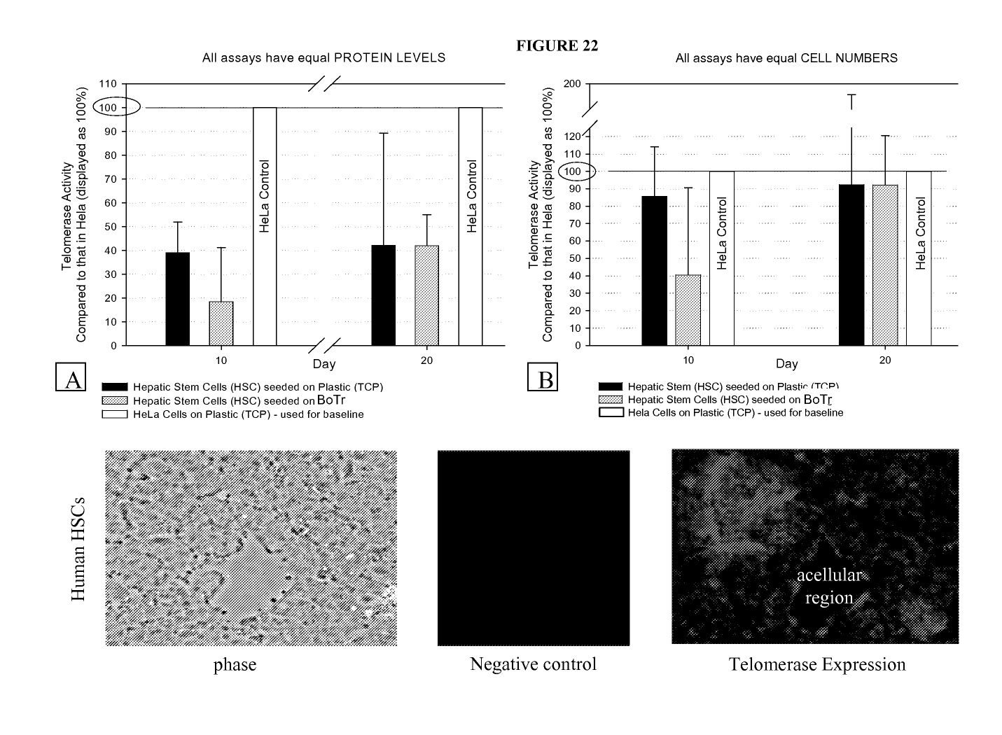

Use of matrix substrata found in abundance in embryonic tissues (e.g., laminin and collagen III and IV) induced specific morphological and functional changes in hepatoblasts and also selected for hepatic stem cells (HSC), the precursors to hepatoblasts. FIG. 9 shows the morphology of fetal liver cells seeded on TCP, laminin, collagen III, and collagen IV surfaces and cultured for 3 to 10 days. The 10× images on day 3 are referred to as TCP3, laminin3, collagenIII3, and collagenIV3, whereas the 4× images on day 10 are referred to as TCP10, laminin 10, collagenIII10, and collagenIV10.

Day 3: The control TCP3 colonies, displayed large numbers of hepatoblasts (h) along with small numbers of non-parenchymal (np) cells. Comparatively, the day 3 cultures on laminin consisted of predominantly hepatoblasts but with fewer cells attached and increased cell spreading. In contrast, the cells on collagen III selected for the hepatic stem cells. Colonies of hepatic stem cells were recognizable as tightly packed cells with uniform morphology and with doubling times of 1.2 days. The cells had characteristic expression of HSC specific antigens (e.g., EpCAM+, NCAM+, albumin+, AFP−, CK19+, CK8+ and 18+). Additionally, HSC colony cells had characteristic diameters ranging from 7-9 mm along with nuclei that occupy most of the cell cytoplasm. The colony cells materialized around day 3 and remained surrounded by hepatoblasts. As well, liver cells seeded on collagen IV displayed many hepatoblasts throughout the culture surface along with beginning developments of two small HSC colonies.

Day 10: The micrographs of the Day 10 results were taken, at 4× magnification to facilitate imaging the entirety of some HSC colonies. For TCP10 control cultures, a small HSC colony developed and remained surrounded by large numbers of hepatoblasts. This small colony acquired a distinct outer thick ridge with convex middle. For the laminin10 cultures, hepatoblasts aggregated into small tightly bound aggregates forming 3-dimensional structures with spherical arrangements and thick tissue layers inside small diameter colonies. The collagenIII10 cultures contained large amounts of ‘flatten and spread’ HSC colonies maintained as tightly packed cell-cell interactions that seemingly precluded the presence of other cell types. In the micrograph shown, few hepatoblasts subsisted between HSC proliferating colonies. Lastly, the collagenIV10 cultures contained both hepatoblasts and a newly arising HSC colony with distinctive colony borders and raised border ridges.

Further detailed investigations of HSC colonies are shown in FIG. 10. In these figures, the attachment substratum was Collagen IV coated at 4.15 μg/cm², and the hepatic stem cell colony was imaged at day 12. As shown, few other cell phenotypes were observed in the vicinity of this colony or elsewhere in the micrograph. Thus, this environment may be considered to select for particular cell types. Prior to acquiring the 10× micrograph shown in FIG. 10A, the HSC colony was visually monitored through days 4-11. During this time, the tightly compacted HSCs originated as small aggregates of less than about 10 cells and proliferated into a larger colony containing hundreds to thousands of cells as shown within the highlighted circular area labeled in FIG. 10A.

On day 12 and within an 8-hour time period, however, two significant “eruptions” or outgrowths of differentiated cells occurred at the HSC colony edges resulting in cells with antigenic and morphological profiles typical of hepatoblasts. These outgrowths were distinguishable as loosely-packed, differentiated cells with larger diameter and distinctive channels (bile caniliculi). FIG. 10B displays a 20× micrograph with central focus on the cell outgrowths. In this figure, the outgrowth of hepatic progenitor cells had diameters ranging between 15 and 21 nm, increased cytoplasmic to nucleus ratios, a single nucleus, and cell-cell contact sustained with definitive cell borders and extracellular matrix separation. Additionally, morphological tracking of differentiated cells emerging from HSC colonies showed about 1200 new cells during the first 8-hours of this expansion.

Matrix components within the periportal zone and in the liver's stem cell niche are distinct from those found in association with the mature parenchymal cells and elicit distinct biological responses from purified subpopulations of human hepatic stem/progenitor cells. These differences are likely to provide diverse signals that modify cell responses and activate dynamic expressions. By determining how distinct classes of extracellular matrix components induce in vivo and in vitro cell activities, microenvironments can be reproduced in vitro to expand and differentiate HSC populations for the replacement or repopulation of diseased tissues.

In this way, transplanted cells obviate whole organ replacement all together. Furthermore, in vitro devices such as bioreactors may be seeded with hepatic progenitors enveloped in an appropriate extracellular matrix and soluble signaling environment so they populate device subcompartments with viable tissue structures. In this way, bioartificial devices can be utilized for pharmacology studies, vaccine developments, and as a bridge between organ failure and organ transplantation. Indeed, the results obtained from these investigations suggest that utilizing these cells may be an avenue to improve cell sourcing limitations that currently inhibit both cell therapy and bioreactor device medical treatments options.

While the invention has been described in connection with specific embodiments thereof, it will be understood that it is capable of further modifications and this application is intended to cover any variations, uses, or alterations of the invention following. In general, the principles of the invention and including such departures from the present disclosure as come within known or customary practice within the art to which the invention pertains and as may be applied to the essential features hereinbefore set forth and as follows in the scope of the appended claims.

We claim:

1. A method of propagating hepatic progenitors in vitro comprising:
(a) providing isolated hepatic progenitors and
(b) culturing the isolated hepatic progenitors on one or more extracellular matrix component found in the stem cell compartment of liver.

2. The method of claim 1 in which the extracellular matrix component found in the stem cell compartment of liver is selected from group consisting of a type III collagen, a type IV collagen, a laminin, a hyaluronan or a combination thereof.

3. The method of claim 2 in which the extracellular matrix component found in the stem cell compartment of liver is a type III collagen or a type IV or a combination thereof.

4. The method of claim 2 in which hepatic progenitors are further cultured on an extracellular matrix component selected from the group consisting of a type III collagen, a basal adhesion molecule, a proteoglycan (PG), a glycosaminoglycan heparan sulfate, elastin an combinations thereof.

5. The method of claim 4 in which the basal adhesion molecule is fibronectin.

6. The method of claim 4 in which the PG is heparan sulfate-PG, chondroitin sulfate PGs or a combination thereof.

7. The method of claim 4 in which the glycosaminoglycan is a heparan sulfate, a heparin, a chondroitin sulfate, a dermatan sulfate, a hyaluronans or a combination thereof.

8. The method of claim 2 in which the extracellular matrix components are type III collagen and laminin.

9. The method of claim 1 in which the isolated hepatic progenitors are isolated hepatic stem cells, isolated hepatoblasts, committed hepatic progenitors, or a combination thereof.

10. The method of claim 9 in which the isolated hepatic progenitors are isolated hepatic stem cells.

11. The method of claim 1 in which the hepatic progenitors are further cultured in the presence of feeder cells.

12. The method of claim 11 in which the feeder cells are embryonic or fetal.

13. The method of claim 12 in which the feeder cells are angioblasts or hepatic stellate precursor cells.

14. The method of claim 11 In which the feeder cells derive from any mammalian tissue.

15. The method of claim 11 in which the feeder cells derive from the same species as the hepatic progenitors.

16. The method of claim 1 in which the feeder cells are murine.

17. The method of claim 16 in which the feeder cells are STO feeder cells.

18. The method of claim 1 further comprising serum-free culture medium.

19. The method of claim 1 in which the hepatic progenitors are obtained from adult liver.

20. The method of claim 19 in which the adult liver is adult human liver.

21. The method of claim 2 in which the laminin is at a concentration between about 0.1 to about 10 μg/cm².

22. The method of claim 21 in which the laminin is at a concentration between about 0.5 to about 5 μg/cm².

23. The method of claim 22 in which the laminin is at a concentration of about 0.5 μg/cm².

24. The method of claim 22 in which the laminin is at a concentration of about 1 μg/cm².

25. The method of claim 2 in which the type III or IV collagens are individually at a concentration between about 0.1 to about 15 μg/cm².

26. The method of claim 25 in which the type III or IV collagens are individually at a concentration between about 0.5 to about 8 μg/cm².

27. The method of claim 25 in which the type III or IV collagens are at a concentration between about 1 to about 7 μg/cm².

28. A method of propagating hepatic progenitors comprising:
(a) providing a first layer of comprising a first extracellular matrix component found in the stem cell compartment of liver;
(b) providing a second layer comprising a second extracellular matrix component found in the stem cell compartment of livers; and
(c) culturing isolated hepatic progenitors between the first and the second layers.

29. A container for propagation of hepatic progenitors comprising:
(a) a container, and
(b) an insoluble material comprising at least one extracellular matrix component found in the stem cell compartment of livers;
wherein the insoluble material substantially coats at least one surface of the container.

30. The container of claim 29 in which the container is a tissue culture plate, a bioreactor, a lab cell or a lab chip.
